(19) United States
(12) Patent Application Publication
(10) Pub. No.: US 2020/0224283 A1
(43) Pub. Date: Jul. 16, 2020

(54) RATIOMETRIC BIOSENSOR TO MEASURE INTRACELLULAR NADH/NAD+ REDOX

(71) Applicant: Wisconsin Alumni Research Foundation, Madison, WI (US)

(72) Inventors: Srivatsan Raman, Middleton, WI (US); Yang Liu, Madison, WI (US); Robert Landick, Madison, WI (US)

(21) Appl. No.: 16/739,343

(22) Filed: Jan. 10, 2020

Related U.S. Application Data
(60) Provisional application No. 62/790,764, filed on Jan. 10, 2019.

(51) Int. Cl. C12Q 1/6897; C12N 15/10

(52) U.S. Cl. CPC C12Q 1/6897; G01N 2500/10; G01N 2333/32; C12N 15/1086

(57) ABSTRACT

Described herein is a redox biosensor system for measurement of NADH/NAD⁺ ratio including a host strain or cell-free system including first and a second expression cassette; the first expression cassette including, in operable communication, a constitutive promoter and a gene encoding a Rex allosteric transcription factor that regulates gene expression by binding to NAD(H); and the second expression cassette including, in operable communication, a promoter regulated by the Rex allosteric transcription factor, and a gene encoding a reporter protein, wherein the first expression cassette expresses the Rex allosteric transcription factor, and, in the presence of NAD(H), the Rex allosteric transcription factor drives expression of the reporter protein from the second expression cassette, and wherein the expression of the reporter protein is proportional to the NADH/NAD⁺ ratio.

### CROSS-REFERENCE TO RELATED APPLICATIONS

This application claims priority to U.S. Provisional Application 62/790,764 filed on Jan. 10, 2019, which is incorporated herein by reference in its entirety.

This invention was made with government support under DE-FC02-07ER64494 awarded by the US Department of Energy. The government has certain rights in the invention.

### FIELD OF THE DISCLOSURE

The present disclosure is related to biosensors for the detection of NADH/NAD+ ratio and their methods of use.

### BACKGROUND

Prokaryotic and eukaryotic cells use NAD(H) as a major electron carrier to power hundreds of essential redox reactions, and to directly or indirectly regulate biochemical processes, such as enzyme allostery and post-translational modifications. NAD(H) is highly connected in the metabolic network, and even small perturbations to NADH/NAD+ ratio can propagate widely across cellular processes. Because NAD(H) is an important metabolic marker, observing differences in redox can help distinguish normal vs. diseased cells, estimate chemical potential within different organelles, and identify cell cycle states. In humans, abnormalities caused by an imbalance of NADH/NAD⁺ have been implicated in aging, diabetes, epilepsy and cancer. In bacteria, NADH/NAD⁺ metabolism plays a key role in adaptability to an environment, and community structure.

E. coli has served as an excellent model system to elucidate the basic biology of redox state. Glucose oxidation reduces NAD⁺ to NADH through glycolysis and the TCA cycle. To maintain redox balance for cell survival, NADH is oxidized back to NAD⁺. Anaerobically grown E. coli regenerates NAD⁺ through many fermentative pathways by reducing metabolic intermediates such as pyruvate with NADH when no other electron acceptors (e.g., nitrate) are present. Under aerobic conditions, E. coli utilizes the respiratory chain to oxidize NADH to NAD⁺, and channels the redox energy to generate a proton gradient for ATP synthesis. The E. coli aerobic respiratory chain contains two NADH dehydrogenases (Ndh-I and Ndh-II) and three cytochrome oxidases (Cyt-bo3, Cyt-bd-I and Cyt-bd-II). Genetic and biochemical characterization of these enzyme complexes suggest that they function under different physiological conditions. However, the contribution of aerobic respiratory chain components toward NAD(H) metabolism has not been systematically and quantitatively characterized. The impact of removing respiratory chain enzymes on NADH/NAD⁺ ratio is poorly understood due to redundancies and compensatory roles among them. A broad goal is to gain a systems-level understanding of how deletions across the genome affect redox imbalance. Since redox state is an emergent, global property that is affected by multiple metabolic pathways, a high-throughput genome-wide survey of genetic factors, beyond known candidates in central metabolism, would aid comprehensive understanding of determinants of intracellular redox state. Genome-wide redox screens are also useful in metabolic engineering for optimizing redox state to drive new biosynthesis reactions.

The use of genetically-encoded biosensors has revolutionized microbial genetics and biomanufacturing by enabling high-throughput screening of cellular phenotypes. A genetically-encoded biosensor is usually an allosteric protein that upon binding to a target metabolite, undergoes chemical, conformational or regulatory change coupled to a spectroscopic response. Since each cell is able to ‘self-report’ the concentration of the metabolite, this approach can be scaled to assay thousands of mutants that can be classified in a cell sorter based on the metabolite level. Biosensors exist for several common intracellular metabolites including calcium, cyclic-GMP, ATP and hydrogen peroxide. In contrast, traditional techniques for measuring redox are low-throughput, lack accuracy, or both. Current NAD(H) quantification relies on NADH auto-fluorescence, enzymatic assays, or LC-MS (liquid chromatography coupled mass spectrometry). These methods have three major drawbacks. First, the signal-to-noise is poor due to high background auto-fluorescence. Second, the measurement may be inaccurate owing to the unstable nature of NADH during sample preparation. Third, these assays are cumbersome and time-consuming, which limits the number of variant backgrounds or environmental conditions (e.g., different carbon sources) that can be studied.

What is needed are new biosensors for NAD(H) detection and quantification.

### BRIEF SUMMARY

In one aspect, a redox biosensor system for measurement of NADH/NAD⁺ ratio comprises

a host strain or cell-free system comprising a first expression cassette and a second expression cassette,

the first expression cassette comprising, in operable communication, a constitutive promoter and a gene encoding a Rex allosteric transcription factor that regulates gene expression by binding to NAD(H), and

the second expression cassette comprising, in operable communication, a promoter regulated by the Rex allosteric transcription factor, and a gene encoding a reporter protein,

wherein the first expression cassette expresses the Rex allosteric transcription factor, and, in the presence of NAD(H), the Rex allosteric transcription factor drives expression of the reporter protein from the second expression cassette, and wherein the expression of the reporter protein is proportional to the NADH/NAD⁺ ratio.

In another aspect, a method of determining NADH/NAD⁺ ratio comprises contacting a sample suspected of containing NADH/NAD⁺ with the redox biosensor system described above.

In yet another aspect, a method of screening population of engineered microbes for a target NADH/NAD⁺ ratio comprises contacting the population of engineered microbes with the redox biosensor system described above, and identifying a sub-population of engineered microbes having the target NADH/NAD⁺ ratio.

### BRIEF DESCRIPTION OF THE DRAWINGS

FIG. 1 shows a multiple sequence alignment of B-Rex operator sites from B. subtilis. The shading marks bases that differ across operators and ‘X’s represent a spacer between the half sites.

FIG. 2 shows a structural model of interactions between the major groove helix of B-Rex with the right half site of the perfect palindrome. B-Rex sidechains are modeled in the T-Rex structure. Dashed lines represent hydrogen bonds between B-Rex and the operator.

FIG. 3 shows a structural model of interactions between the major groove helix with the right half site of the imperfect palindrome. Dashed lines represent hydrogen bonds between B-Rex and the operator.

FIG. 4 shows a comparison of redox biosensor-reporter activity of an E. coli promoter embedded with Rex operators containing perfect (ROP-PP) and imperfect (ROP-IP) palindromes. Included is a schematic of the dual-plasmid system. Cells are transformed with pB-Rex expressing B-Rex from a constitutive E. coli promoter and reporter plasmids containing engineered promoters either pROP-PP or pROP-IP driving expression of fluorescent protein.

FIG. 5 shows biosensor properties of pROP-PP. pB-Rex- denotes cells constitutively expressing FbFP from an unregulated promoter at low and high NADH. pB-Rex+ denotes cells co-expressing B-Rex with corresponding promoters to regulate FbFP. The images below the barchart show culture under UV.

FIG. 6 shows the biosensor properties of pROP-IP.

FIG. 7 shows a kit based NAD(H) quantification for aerobic and anaerobic grown E. coli. FU is fluorescent units of FbFP. The error bar is standard deviation of three replicates.

FIG. 8 shows an illustration of respiratory chain enzymes embedded in the inner membrane of E. coli. Ndh-I and II are NADH dehydrogenases, and Cytbo3, Cytbd-I and Cytbd-II are cytochrome oxidases. UQH2 and UQ are the reduced and oxidized forms of quinone (B-D).

FIG. 9 shows GFP reporter fluorescence of wildtype and NADH-dehydrogenase mutants. For each strain, the fluorescence of reporter only (pB-Rex-, left bar) and reporter with B-Rex (pB-Rex+, right bar) are shown. The numbers above the bars represent fold change in biosensor-reporter fluorescence, relative to wildtype. The Y-axis is normalized fluorescence units.

FIG. 10 shows GFP reporter fluorescence of cytochrome oxidase single mutants.

FIG. 11 shows GFP reporter fluorescence of cytochrome oxidase double mutants.

FIG. 12 shows NADH/NAD+ ratio measured with an enzymatic-kit based assay of wildtype and NADH-dehydrogenase mutants. The error bar represents standard deviation of three replicates.

FIG. 13 shows NADH/NAD+ ratio measured with an enzymatic-kit based assay of cytochrome oxidase single mutants.

FIG. 14 shows NADH/NAD+ ratio measured with an enzymatic-kit based assay of cytochrome oxidase double mutants.

FIG. 15 shows the redox state of wildtype E. coli grown on different carbon sources. Fluorescence of reporter only (pB-Rex-, left) and reporter with B-Rex (pB-Rex+, right bar) is shown. The numbers above the bars represent the fold change in biosensor-reporter fluorescence relative to cells grown in glucose. The Y-axis is normalized GFP fluorescence units. The error bar represents standard deviation of three replicates.

FIG. 16 shows an evaluation of a B-Rex biosensor-reporter as a high-throughput screening tool. The X-axis is the proportion of wildtype to mutant in the starting population. The Y-axis is the proportion of wildtype to mutant by PCR genotyping 30 colonies after enrichment using fluorescence-activated cell sorting. Black, gray and light gray bars denote wildtype, and Δndh mutant and ΔappBΔcydB double mutant, respectively.

The above-described and other features will be appreciated and understood by those skilled in the art from the following detailed description, drawings, and appended claims.

### DETAILED DESCRIPTION

A genetically-encoded biosensor for NADH/NAD⁺ ratio in bacteria to facilitate high-throughput redox screens has been developed. The sensor is based on Rex, an allosteric transcription factor that regulates gene expression by binding to NAD(H). Rex undergoes a conformational change upon binding to NADH and several studies have taken advantage of this property of Rex to build NADH/NAD⁺ biosensors by conformational coupling of a fluorescent protein to Rex. Conformational coupling enables rapid response to NADH/NAD⁺ changes, making these biosensors an excellent tool for imaging redox changes in real time. However, new conformational biosensors must be specifically engineered for different fluorescent reporters. For instance, current conformational biosensors engineered for oxygen-dependent fluorescent proteins cannot be used in hypoxic or anaerobic conditions. Further, conformational biosensors are not suitable for regulation of engineered circuits in response to redox changes. Conformational biosensors have not been used for screening variant libraries, possibly due to noisy or uneven signal at steady state. In contrast, regulatory biosensors can interface with endogenous pathways to control engineered circuits, and have been widely used for screening large variant libraries in metabolic engineering. Regulatory biosensors offer several advantages because they can transcriptionally control any gene of choice. Since transcription generates a steady signal, even if the physiology of the cell changes during screening, as it does while cell sorting, the reporter records the state of the cell when the reporter is expressed, and not when it is being read. This is particularly important because bacteria rapidly change redox levels when cells are not continuously exposed to oxygen. Further, a reporter can be chosen to suit experimental conditions (e.g., aerobic vs. anaerobic) or multiple reporters can be used simultaneously for different metabolites. Recently, yeast transcription factor Yap1p was repurposed as a regulatory redox biosensor in S. cerevisiae to select for S. cerevisiae variants with higher NADPH capacity. However, to the best of our knowledge a regulatory NADH/NAD+ biosensor has not been implemented in bacteria to date.

Described herein is a regulatory redox biosensor in E. coli for NADH/NAD⁺ ratio made by porting Rex from B. subtilis (B-Rex) (Uniprot ID: 005521; SEQ ID NO: 1) into E. coli. B-Rex homologs from other microbes including, but not limited to, Staphylococcous aureus and Streptomyces coelicolor can be used. B-Rex was functionalized in E. coli by placing Rex operator sites with different affinities for B-Rex into a constitutive E. coli promoter. To compare different promoter designs, biosensor-reporter activity in aerobic (low NADH) and anaerobic (high NADH) conditions was evaluated, and an optimal design was chosen with good dynamic range and high reporter signal at full induction. Low and high NADH are relative to E. coli grown in rich medium conditions where the NADH is low. Mutations or alternative growth conditions can increase or decrease NADH. To validate the reporter for use in high-throughput screens, for example, the redox biosensor-reporter was applied to investigate the functional contribution of aerobic respiratory chain enzymes on NADH/NAD⁺ redox balance. Without being held to theory, it was hypothesized that knocking out components in the respiratory chain pathway (NADH dehydrogenases and cytochrome oxidases) would abolish or attenuate NADH oxidation, leading to a buildup of NADH. Five of the nine single and double knock outs elevated NADH signal greater than 3-fold over wildtype E. coli. Highest NADH signal (6.1-fold increase) was measured in a strain in which both NADH dehydrogenases were disabled by the deletion of NADH dehydrogenase-II and the F-subunit of NADH dehydrogenase-I. Since redox state can also be altered by changing the carbon source, NADH/NAD⁺ of E. coli grown on several common carbon sources was also tested. Cells grown on acetate show nearly twice the NADH/NAD⁺ signal as glucose, which is consistent with a previous metabolomics study. The utility of the Rex biosensor-reporter for high-throughput pooled screening was demonstrated by enriching high NADH cells at an abundance of 1 in 10,000 from a mixed population by fluorescence activated cell sorting.

In an aspect, a redox biosensor system for measurement of NADH/NAD⁺ ratio comprises

a host strain or cell-free system comprising a first and a second expression cassette,

the first expression cassette comprising, in operable communication, a constitutive promoter and a gene encoding a Rex allosteric transcription factor that regulates gene expression by binding to NAD(H), and

the second expression cassette comprising, in operable communication, a promoter regulated by the Rex allosteric transcription factor, and a gene encoding a reporter protein,

wherein the first expression cassette expresses the Rex allosteric transcription factor, and, in the presence of NAD(H), the Rex allosteric transcription factor drives expression of the reporter protein from the second expression cassette, and wherein the expression of the reporter protein is proportional to the NADH/NAD⁺ ratio.

“Operably linked” means that the nucleotide sequence of interest is linked to the regulatory sequence(s) in a manner that allows for expression of the transcription factor or the reporter gene, for example. The constitutive and regulated promoters can be operably linked in any location, or multiplicity, so long as they are operably linked to allow for expression of the operably linked gene. Exemplary reporter proteins include GFP and FbFP (anaerobic condition).

Advantageously, the redox biosensors described herein are functional under hypoxic, aerobic or anaerobic conditions.

In an embodiment, the constitutive promoter in the first expression cassette comprises E. coli promoter J23101, (AGCTAGCTCAGTCCTAGGTATTATGCTAGC; SEQ ID NO: 2), apFAB54 (GGCGCGCCTTGACATAAAGTCTAACCTATAGGATACTTACAGCCATACAA; SEQ ID NO: 3), BBa_J2310 (GGCGCGCCTTGACGGCTAGCTCAGTCCTAGGTACAGTGCTAGCTTAAT; SEQ ID NO: 4), apFAB338 (GGCGCGCCTTGACAATTAATCATCCGGCTCCTAGGATGTGTGGAGGGA; SEQ ID NO: 5), apFAB323 (GGCGCGCCTTGTATTTTAATCATCCGGCTCGTATAATGTGTGGAATCCA; SEQ ID NO: 6); BBa_J23119 (GGCGCGCCTTGACAGCTAGCTCAGTCCTAGGTATAATGCTAGCACGA; SEQ ID NO: 7); and apFAB150 (GGCGCGCCCACGGTGTTAGACATTTATCCCTTGCGGCGATATAATGTGTGGATC CAT; SEQ ID NO: 8).

In certain embodiments, the promoter regulated by the Rex allosteric transcription factor comprises a modified constitutive promoter comprising one or more Rex operator sites.

The first and second expression cassettes can be present on the same or a different nucleic acid molecule, such as the same or different plasmids or expression cassettes. For example, the first expression cassette is present in a high or low copy number sensor plasmid, and the second expression cassette is present on a reporter plasmid.

In a specific embodiment, the Rex allosteric transcription factor is B. subtilis Rex.

Exemplary host strains are typically prokaryotes. In an embodiment, the host strain comprises organisms of the genus Escherichia, Bacillus, Staphylococcus, Caulobacter, Streptococcus, Streptomyces, Mycoplasma, Aliivibrio, Synechocystis, Azotobacter, Pseudomonas, Agrobacterium, Zymomonas, or a combination comprising at least one of the foregoing. Host strains and examples are provided in Table 1:

TABLE 1
Organism genera and example species

| Organism genus | Example species |
|---|---|
| Escherichia | E. coli |
| Bacillus | B. subtilis, B. thermophiles, B. anthracis |
| Staphylococcus | S. aureus. S. epidermidis |
| Caulobacter | C. Crescentus |
| Streptococcus | S. pyogenes, S. pneumoniae |
| Thermus | T. aquaticus, T. thermophilus |
| Streptomyces | S. coelicolor, S. antibioticus, S. avermitilis |
| Mycoplasma | M. Pneumoniae, M. genitalium |
| Aliivibrio | A. fischeri |
| Synechocystis | S. sp PCC6803 |
| Azotobacter | A. vinelandii |
| Pseudomonas | P. aeruginosa, P. putida, P. syringae |
| Agrobacterium | A. tumefaciens |
| Zymomonas | Z. mobilis |

A specific host strain is E. coli.

In an embodiment, wherein the dynamic range of the biosensor is the ratio of NADH to NAD+ in fully induced vs. uninduced states.

Exemplary reporter proteins include GFP and FbFP.

Also included herein are liquid-based assays, paper strips, or multi-well plate assays (96 well or 384 well plates, for example) comprising the biosensors described herein.

Also included herein are methods of determining NADH/NAD⁺ ratio in engineered microorganisms and methods of screening populations of engineered microbes for a target NADH/NAD⁺ ratio.

Many commercial products that are difficult to produce synthetically are today produced by fermenting organisms, such as microbes engineered for such purpose. Such products include alcohols (e.g., butanol, ethanol, methanol, 1,3-propanediol); organic acids (e.g., acetic acid, citric acid, gluconate, gluconic acid, itaconic acid, lactic acid, succinic acid, 2,5-diketo-D-gluconic acid); ketones (e.g., acetone); amino acids (e.g., glutamic acid); gases (e.g., H₂ and CO₂), and more complex compounds, including, for example, antibiotics (e.g., penicillin and tetracycline); enzymes; vitamins (e.g., riboflavin, B₁₂, beta-carotene); and hormones. Biofuels and biochemicals, such as ethanol and lactate, are produced by fermentation, particularly under oxygen-limited conditions.

Woody biomass such as agricultural residues, wood chips, municipal solid wastes, and paper wastes can be transformed into bio-ethanol and bio-diesel. Thermophilic bacteria, for example, have been engineered to produce ethanol from the cellulose and/or hemicellulose fractions of biomass. Examples of such thermophilic bacteria include Clostridium thermocellum and Thermoanaerobacterium saccharolyticum, among others.

The biosensors described herein can also be used for measure redox of cells to study disorders associated with metabolism including cancer and autoimmune diseases.

In an embodiment, a method of screening a population of engineered microbes for a target NADH/NAD⁺ ratio comprises contacting the population of engineered microbes with the redox biosensor system as described herein, and identifying a sub-population of engineered microbes having the target NADH/NAD⁺.

Multiple rounds of screening can be performed. Thus, in an embodiment, the method further comprises contacting the sub-population of engineered microbes having the target NADH/NAD⁺ ratio with the redox biosensor system, identifying a further sub-population of engineered microbes having the target NADH/NAD⁺ ratio, and optionally repeating the contacting and identifying for a plurality of rounds to provide an optimized sub-population of engineered microbes having the target NADH/NAD⁺ ratio.

The methods can further comprise deep sequencing the sub-population of engineered microbes having the target NADH/NAD⁺ ratio. The method can optionally further comprise identifying one or more genes in the sub-population of engineered microbes that perturb redox state.

The invention is further illustrated by the following non-limiting examples.

### EXAMPLES

### Methods

Materials: Q5® HiFi DNA polymerase, OneTaq® DNA polymerase and NEBuilder® HiFi DNA assembly mix were purchased from New England Biolabs. Wizard® plus SV Minipreps DNA Purification System and NAD/NADH-Glo® Assays were purchased from Promega. NADH and NAD were purchased from Roche. MOPS minimal buffer (5×) was purchased from Teknova.

Structural comparison of Rex operators: Structural analysis was based on Thermus aquaticus Rex (PDB: 3ikt) using Pymol. For visualizing the Rex interaction with the operator, the DNA sequence was modeled in using 3DNA and then visualized with Pymol.

Strains and growth conditions: All strains used in this study were derivatives of E. coli K12 RL3000 (see Table 2). Respiratory chain mutants of RL3000 were constructed with P1 transduction using Keio collection, followed by marker removal by PCP20 plasmid. Overnight cultures of wildtype or mutants grown in LB were inoculated into MOPS minimal media with glucose or other carbon sources at a starting OD600 of approximately 0.05. To compare biosensor-reporter induction in anaerobic condition vs aerobic conditions, cells were cultured with MOPS minimal media with 20 mM glucose. RL3000 wild-type strain was grown well aerated in 5 mL media in 125 mL flasks, and shaking at 250 rpm for aerobic growth. RL3000 was cultured in 5 mL media in 125 mL flasks, stirred by magnetic bars in an anaerobic chamber for anaerobic growth. MOPS minimal media with 20 mM glucose was used in the study of wildtype and respiratory chain mutants. For studying the effects of carbon sources, MOPS minimal media with 20 mM glucose was used as a reference; concentrations of other carbon sources were adjusted to match the same mole of carbons as glucose (for example, 40 mM glycerol) and cells were grown aerobically as described above.

TABLE 2
Strains in this study

| strain name | Description | Source |
|---|---|---|
| RL3000 | MG1655 ilvG+ rph+ rfb-50 yciI::82bp | Ghosh, I. N.; Landick, R. |
| | nudF(G102V) Δ(glcByghO) | OptSSeq: High-Throughput |
| | crl+(Δins1-I) ybhJ(L54I) ychE+(Δins5- | Sequencing Readout of |
| | U) mntP(G25D) | Growth Enrichment |
| | yecD(N186H) gatC+ glpR+ | Defines Optimal Gene |
| | flhD+(ΔinsAB-5) | Expression Elements for |
| | | Homoethanologenesis. ACS |
| | | Synth. Biol. 5, pp. 1519- |
| | | 1534. 2016. |
| Δndh | RL3000 Δndh:FRT | This work |
| ΔnuoF | RL3000 ΔnuoF:FRT | This work |
| ΔndhΔnuoF | RL3000 Δndh:FRT, ΔnuoF:kanR | This work |
| ΔcyoB | RL3000 ΔcyoB:FRT | This work |
| ΔappB | RL3000 ΔappB:FRT | This work |
| ΔcydB | RL3000 ΔcydB:FRT | This work |
| ΔcyoBΔappB | RL3000 ΔcyoB:FRT, ΔappB:kanR | This work |
| ΔcyoBΔcydB | RL3000 ΔcyoB:FRT, ΔcydB:kanR | This work |
| ΔappBΔcydB | RL3000 ΔappB:FRT, ΔcydB:kanR | This work |

Plasmid construction: Plasmids are listed in Table 3 and primers in Table 4. Q5® HiFi DNA polymerase (NEB) was used for PCR. Plasmids were constructed using Gibson assembly with gel purified PCR products. After transforming chemically-competent E. coli DH5α with Gibson assembly products, cells were recovered and plated on LB plates with corresponding antibiotics. Plasmids from single colonies were harvested after overnight growth with antibiotics and sequenced for validating the clone (Quintarabio Biosciences).

TABLE 3
Plasmids

| Plasmids | Descriptions | Source |
|---|---|---|
| pBBR1-MCS5 | Plasmid derived from the plasmid | Kovach, M. E.; Elzer, P. H.; |
| | pBBR with the cryptic pBBRori | Steven Hill, D.; Robertson, |
| | encoding gentamycin resistance; | G. T.; Farris, M. A.; Roop, |
| | aacC1(GnR)-Plac-lacZα-MCS-[mob- | R. M.; Peterson, K. M. Four |
| | rep](pBBRori) | New Derivatives of the |
| | | Broad-Host-Range Cloning |
| | | Vector PBBR1MCS, |
| | | Carrying Different |
| | | Antibiotic-Resistance |
| | | Cassettes. Gene, 166, pp. |
| | | 175-76, 1995. |
| pBAsyn | Ethanol fermentation pathway plasmid | Ghosh, I. N.; Landick, R. |
| | contain FMN-based Fluorescent Protein | OptSSeq: High-Throughput |
| | | Sequencing Readout of |
| | | Growth Enrichment Defines |
| | | Optimal Gene Expression |
| | | Elements for |
| | | Homoethanologenesis. ACS |
| | | Synth. Biol. 5, pp. 1519-1534, |
| | | 2016. |
| pJKR-L-cdaR | Glutarate biosensor, pSC101 origin | Rogers, J. K; Guzman, C. |
| | | D.; Taylor, N. D.; Raman, S.; |
| | | Anderson, K.; Church, G. M. |
| | | Synthetic Biosensors for |
| | | Precise Gene Control and |
| | | Real-Time Monitoring of |
| | | Metabolites. Nucleic Acids |
| | | Res. 43(15), pp. 7648-7660, |
| | | 2015. |
| pJ251-GERC | Plasmid containing GFP, mCherry, | addgene #47441 |
| | p15A origin | |
| pB-Rex | Rex sensor plasmid contain B-Rex, | This work |
| | pBBR1 orgin, GnR | |
| pROP-PP- | Reporter plasmid contains FbFP with | This work |
| FbFP | perfect palindrome ROP, and mCherry, | |
| | pSC101 origin, SpR | |
| pROP-IP-FbFP | Reporter plasmid contains FbFP with | This work |
| | imperfect palindrome ROP, and | |
| | mCherry, pSC101 origin, SpR | |
| pROP-IP-GFP | Reporter plasmid contains GFP with | This work |
| | imperfect palindrome ROP, and | |
| | mCherry control, pSC101 origin, SpR | |

TABLE 4
Primers

SEQ

| | | ID |
|---|---|---|
| To generate pB-Rex | | NO: |
| pRM914_R_ | AGGCGCGCCCGGGATCTCGACGCTCTCCCT | 9 |
| sensor | TATGAGATCT | |
| Rexsensor_F | GCTTGATATCGAATTCCTGC | 10 |
| BsRex600_F1 | CAGACGATTCTATTACAAATCCAGAATGAA | 11 |
| | TAAGGATCAATCAAA | |
| BsRex600_F2 | CCTAGGATGTGTGGAGGGACGACTAACACC | 12 |
| | AGACGATTCTATTACAA | |
| apFAB338 | TCGAGATCCCGGGCGCGCCTTGACAATTAA | 13 |
| | TCATCCGGCTCCTAGGATGTGTGGAGGGAC | |
| To generate pROP-PP-FbFP or pROP-IP-FbFP | | |
| Bsevo_F1 | AATCTATTAGGAGGTTTATAGCATGGCGTC | 14 |
| | CTTTCAGTCT | |
| Bsevo_short_R | AGGAATTCGATATCAAGCTTTTACTCCAAC | 15 |
| | AGCTTCTCGT | |
| GAreporter_F | AAGCTTGATATCGAATTCCTGCAGC | 16 |
| R_GA_common | GCTAGCATCTCGAGGTGAAGAC | 17 |
| reporterSC101_ | CCCTTTTTCTTTAAAACCGAAAAGA | 18 |
| F | | |
| mcherry_ | TGGAGCAAGAAGGTAAGCGAGGATAAGAAG | 19 |
| divergent_F1 | GAAAAGAATGGTTTCCAAGGGCGAG | |
| mcherry_ | CTTTACGGCTAGCTCAGCCCTAGGTATTAT | 20 |
| divergent_F2 | GCTAGCATGGAGCAAGAAGGTAAGC | |
| mcherry_ | TCGGTTTTAAAGAAAAAGGGGGCGCGCCTT | 21 |
| divergent_F3 | TACGGCTAGCTCAGC | |
| mcherry_ | GGCAAGGTGTCACCACCCTGTTATTTGTAC | 22 |
| divergent_R | AGCTCATCCA | |
| reporterSC101_ | TGGATGAGCTGTACAAATAACAGGGTGGTG | 23 |
| R | ACACCTTGCC | |
| ROP_F1 | GATGCTAGCAAAAAAAGAGTATTTACAGCT | 24 |
| | AGCTCAGTCC | |
| ROP_F2 | TTTCGTCTTCACCTCGAGATGCTAGCAAAA | 25 |
| | AAAGA | |
| ROP_R2 | TTTATAAGAATTGTGAATTCTTTTTGTGAA | 26 |
| | GTATTTCACA | |
| pROP-PP primers | | |
| ROP_PP | TTTACAGCTAGCTCAGTCCTAGGTATTATT | 27 |
| | ACAGCCATGTGAAATACTTCACAAAAAGAA | |
| ROP_PP_R1 | TTTATAAGAATTGTGAATTCTTTTTGTGAA | 28 |
| | GTATTTCACA | |

pROP-IP primers

| | | |
|---|---|---|
| ROP_IP | TTTACAGCTAGCTCAGTCCTAGGTATTATT | 29 |
| | ACAGCCATGTGAAATATTGAGCA | |
| ROP_IP_R1 | TATAAGAATTGTGAATTCTTTTTGCTCAAT | 30 |
| | ATTTCACA | |

To generate pROP-IP-GFP

| | | |
|---|---|---|
| F_GA_common | GAATTCATTAAAGAGGAGAAAGGTACCATG | 31 |
| R_GA_common | GCTAGCATCTCGAGGTGAAGAC | 32 |
| mcherry_ | TGGAGCAAGAAGGTAAGCGAGGATAAGAAG | 33 |
| divergent_F1 | GAAAAGAATGGTTTCCAAGGGCGAG | |
| mcherry_ | CTTTACGGCTAGCTCAGCCCTAGGTATTAT | 34 |
| divergent_F2 | GCTAGCATGGAGCAAGAAGGTAAGC | |
| mcherry_ | TCGGTTTTAAAGAAAAAGGGGGCGCGCCTT | 35 |
| divergent_F3 | TACGGCTAGCTCAGC | |
| mcherry_ | GGCAAGGTGTCACCACCCTGTTATTTGTAC | 36 |
| divergent_R | AGCTCATCCA | |
| reporterSC101_ | TGGATGAGCTGTACAAATAACAGGGTGGTG | 37 |
| R | ACACCTTGCC | |
| ROP_F1 | GATGCTAGCAAAAAAAGAGTATTTACAGCT | 38 |
| | AGCTCAGTCC | |
| ROP_F2 | TTTCGTCTTCACCTCGAGATGCTAGCAAAA | 39 |
| | AAAGA | |
| ROP_R2 | TTTATAAGAATTGTGAATTCTTTTTGTGAA | 40 |
| | GTATTTCACA | |
| ROP_IP | TTTACAGCTAGCTCAGTCCTAGGTATTATT | 41 |
| | ACAGCCATGTGAAATATTGAGCA | |
| ROP_IP_R1 | TATAAGAATTGTGAATTCTTTTTGCTCAAT | 42 |
| | ATTTCACA | |

Plasmid pB-Rex was constructed by amplifying the B-Rex gene from B. subtilis genomic DNA and cloned into pBBR1-MCSS under the control of synthetic promoter apFAB338 (gnR). Plasmids pROP-PP and -IP reporters were derivatives of a plasmid with pSC101 origin of replication containing spectinomycin resistance cassette. Additionally, a constitutively expressed mCherry was amplified from pJ251-GERC (a gift from George Church Addgene plasmid #47441) was inserted into the reporter plasmids (pROP-PP and -IP) to serve as a control for normalization for aerobic experiments. FMN-based fluorescent protein (FbFP) was amplified from pBAsyn and assembled with pROP-PP and -IP reporters. GFP was used reporter in pROP-IP in the study of respiratory chain mutants. The ribosome binding Site (RBS) sequence was designed using Salis RBS calculator.

Fluorescence normalization: Cells were grown to an OD600 approximately 0.5 before measuring fluorescence in a Tecan M200 plate reader. The excitation and emission wavelengths used for measuring FbFP, GFP and mCherry are 450 nm/490 nm, 488 nm/530 nm and 580 nm/625 nm, respectively. FbFP fluorescence for comparison of aerobic and anaerobic conditions was normalized by cell density (OD600) and GFP fluorescence for investigating respiratory chain mutants was additionally normalized by mCherry fluorescence. For the experiment investigating effects of carbon sources, fluorescence was normalized to wild-type strain grown in glucose.

Quantification of NAD⁺ and NADH in different strains: NAD⁺ and NADH quantifications were performed according to the NAD/NADH Glo® assay protocol from Promega. E. coli strains were grown to OD approximately 0.5 aerobically in flask or anaerobically in anaerobic chamber as described before. 1 mL cells was spun down and resuspended in 200 μL phosphate-buffered saline. 50 μL of cell suspension was then added to a 96 well plate. 50 μL lysis buffer (0.2 M NaOH and 1% DTAB) were added to the cell suspension to lyse the cells. To measure NADH, 50 μL of the lysed cell suspension was incubated at 60° C. for 15 minutes. Then 50 μL Trizma®/HCl solution (mixing 0.4 M HCl and 0.5 M Trizma® base) was added to the base treated cell suspension after cooling to room temperature for 10 minutes. To measure NAD⁺, 25 μl of 0.4 M HCl was added to 50 μL of lysed cell suspension and then incubated at 60° C. for 15 minutes. After the treatment, 254 of Trizma® based was added to the acid treated cell suspension cooling to room temperature for 10 minutes. Finally, 50 μL of NAD/NADH-Glo® Detection Reagent were mixed with 50 μL of the treated cell suspension in a 96 well plate and the mixture was incubated at room temperature and the luminescence was measured at a 5-minute interval in the Tecan M200 plate reader. 504 NAD⁺ and NADH standards (ranging from 0 to 400 nM) were prepared and mixed with 50 μL of NAD/NADH-Glo® Detection Reagents; and the standard curves were generated and measured at the same time of the samples. The concentrations of NAD⁺ and NADH of each E. coli strain were calculated based on the standard curves of NAD⁺ and NADH. The ratios of NADH and NAD⁺ are reported.

Fluorescence Activated Cell Sorting (FACS): A SONY LE-SH800 was used for cell sorting. All the experiments were performed with biological triplicates. Δndh and wild-type strains containing both pROP-IP and pB-Rex plasmids were grown to OD600 approximately 0.5-0.6. Wild-type and Δndh were adjusted to similar cell densities and then mixed with ratio of wild-type:Δndh at 1:1, 10:1, 100:1, 1000:1 and 10,000:1. The same procedure was carried for mixing ΔappBΔcydB with wild-type. During the cell sorting procedure, the top 5 percent of the population of each mixture was sorted and the sorted cells were then recovered in SOB for 2 h. Cells mixed at ratios of 1:1 and 10:1 were recovered after sorting and plated on LB plates with corresponding antibiotics. Cells mixed at ratios higher than 10:1 were recovered after sorting and cultured in 5 mL MOPS minimal with 20 mM glucose and grown aerobically as described earlier. The top 5% population of the regrown cultures were sorted again, recovered in SOB and plated on LB plates with corresponding antibiotics. To estimate the percentage of mutant cells after sorting, 10 colonies were checked by PCR (3 replicates) using OneTaq® DNA polymerase. PCR products from mutants had a distinct size from wild-type. High NADH mutant recovery was calculated as the fraction of mutants divided among all colonies tested. Average and standard deviation of the recovery were then calculated from biological triplicates. Enrichment was calculated by multiplying the recovery by the corresponding mixing ratio.

### Example 1: Structure-Guided Analysis of Rex Operator Sites

B-Rex was chosen for this study because it is biochemically and structurally well characterized with several known operator sites. B-Rex binds to NADH and NAD+, but is only induced by NADH. Thus, transcription from a B-Rex-regulated promoter is proportional to the NADH/NAD⁺ ratio. To port B-Rex into E. coli, a constitutive E. coli promoter was modified to become B-Rex-responsive by inserting a B-Rex operator site after the transcription start site. Since B-Rex is a repressor, it would block RNA polymerase at low NADH by remaining bound to the operator and enable transcription by dissociating at high NADH to achieve NADH-dependent gene regulation of a downstream fluorescent reporter. This strategy was used over using the native B. subtilis promoter because this approach can be adapted to port B-Rex to other non-model microbes and also allows tuning of biosensor properties in the absence of layered regulation embedded in native promoters. The choice of operator site balanced two biosensor properties—dynamic range and maximum reporter signal. Dynamic range is the ratio of reporter expression at high and low NADH. Maximum reporter signal is the absolute fluorescence units when a biosensor is fully induced. Both dynamic range and maximum reporter signal should be large to accurately measure and resolve small redox differences between cells. High dynamic range improves signal-to-noise discrimination. High maximum reporter signal minimizes false positives from fluorescence-based cell sorting due to better sorting accuracy at higher signal intensities. However, maximizing one property can have an opposing effect on the other. If a biosensor has high affinity for an operator, then the dynamic range would be large due to the low baseline signal but the maximum reporter signal may be small. Alternatively, if a biosensor has low affinity for an operator, then maximum reporter signal would be large but the dynamic range would be small due to high reporter baseline. Therefore, choosing an operator with the right binding affinity provides optimal biosensor-reporter activity.

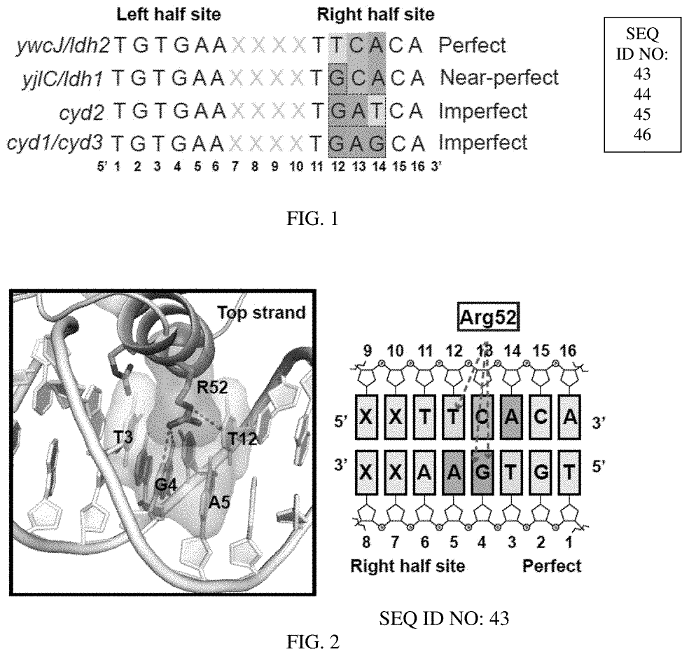

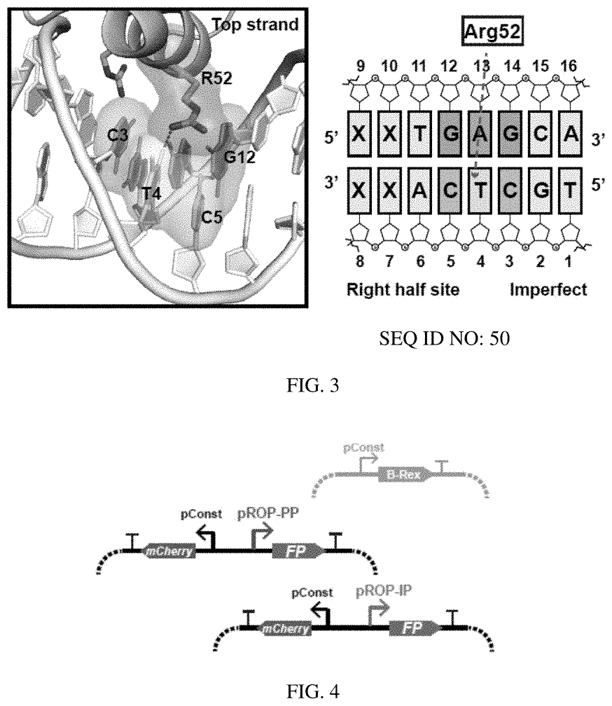

There are seven known B-Rex operator sites (ROP) in B. subtilis, all sharing identical left half-sites, but with different right half-sites. The ROPs were classified into three groups: perfect, near-perfect and imperfect palindromes (FIG. 1), with the near-perfect and imperfect palindromes one and three bases away from a perfect palindrome, respectively. Differences between the ROPs were at base positions 12, 13 and 14 and the spacer between the half-sites (marked as ‘X’s in FIG. 1). Because it was unclear from sequence analysis alone which operator had the optimal affinity for B-Rex, two ROPs with expected tighter and weaker binding were tested. A structure-based approach was used to analyze interactions between the protein and DNA to choose suitable operators. The crystal structure of B-Rex is not complexed with DNA. Therefore, DNA-bound structure of a close homolog from Thermus aquaticus, T-Rex, was chosen for analysis. The DNA-binding domains of T-Rex and B-Rex share a sequence identity of 41% and a backbone root-mean square deviation of 1.24 Å. Six out of the eleven residues (T-Rex F43 to G53) along the major groove helix are identical to B-Rex, and the crucial DNA-contacting residues are conserved or similar (R46, K47 in T-Rex and R51, R52 in B-Rex). The T-Rex-DNA structure shows that the spacer does not directly contact the protein backbone or sidechains, and hence is not likely to contribute to binding. Since the left half-sites are identical, our attention was focused on the variant triplet (bases 12, 13 and 14) in the right half-site as the likely driver of binding affinity differences between operators. Arginine at position 52 was modeled for B-Rex to replace the lysine at position 47 of T-Rex. Arg52 makes strong interactions with the perfect palindrome than the imperfect palindrome. In the perfect palindrome TGTGAAXXXXTTCACA (SEQ ID NO: 43), Arg52 makes extensive hydrogen bonds with G4 and T12 in the right half site. Arg52 interacts with O6 and N7 of base G4, and O4 of base T12 (FIG. 2). Besides the hydrogen bonding interactions, Arg52 is also stabilized by hydrophobic interactions provided by bases T3 and T12. The perfect palindrome (operator in 5′-UTR of ywcJ and ldh2) was chosen as the first candidate due to optimal protein-DNA contacts suggesting tight binding through with hydrogen bonding, hydrophobic contacts, and stacking interactions. The structural model of B-Rex with imperfect palindrome TGTGAAATATTGAGCA (SEQ ID NO: 47) was modeled in by 3DNA (operator in 5′-UTR of cyd1). However, Arg52 loses two hydrogen bonds due to the sequence changes (FIG. 3). Notably, the hydrophobic interactions provided by T3 and T12 in the perfect palindrome are significantly reduced in the imperfect palindrome (FIG. 3). Without being held to theory, it is believed that the imperfect palindrome will likely have lower affinity for B-Rex than the perfect palindrome. Two promoters were constructed: one embedded with the perfect palindrome TGTGAAATACTTCACA (SEQ ID NO: 48) (ROP-PP) and another with an imperfect palindrome TGTGAAATATTGAGCA (SEQ ID NO: 49), (ROP-IP). Both operators were inserted after the transcription start site on a medium strength, constitutive E. coli promoter, J23101 to generate promoter pROP-PP and pROP-IP.

### Example 2: Comparing the Performance of Promoter Designs Under Aerobic Vs. Anaerobic Growth

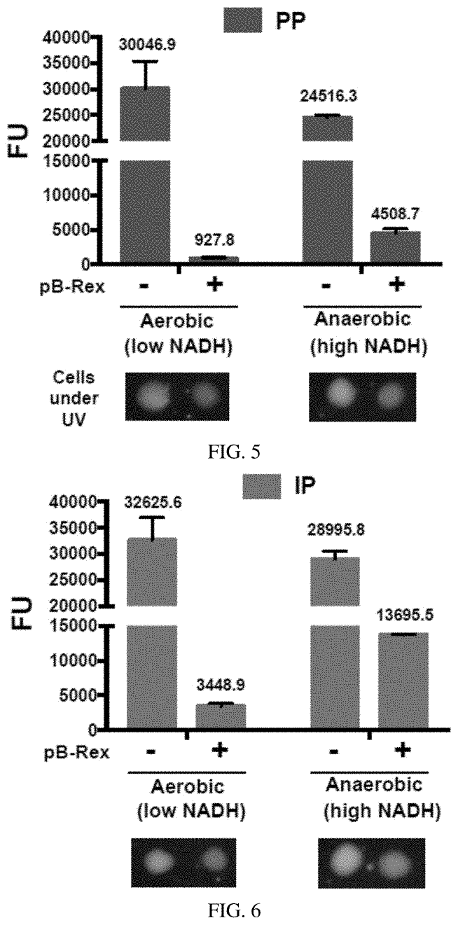

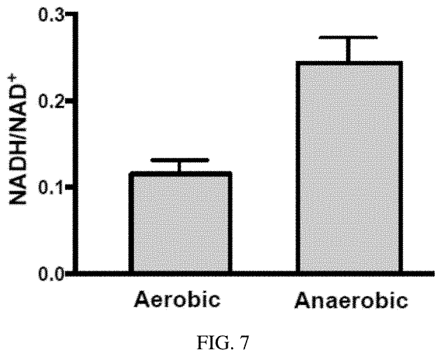

A dual-plasmid system was constructed to evaluate the promoters. One plasmid constitutively expressed B-Rex (pB-Rex) and other carried a fluorescent reporter gene regulated either by pROP-PP or pROP-IP (FIG. 4). The biosensor-reporter system cannot be induced by exogenous NADH in the growth media because NADH is not permeable through the bacterial membrane. To induce the biosensor-reporter, E. coli was grown anaerobically since the NADH/NAD⁺ ratio is higher in E. coli grown anaerobically than aerobically. This result was independently verified using an enzyme-based commercial kit. The performance of ROP-PP and ROP-IP promoter designs was evaluated by comparing reporter fluorescence of cells grown in aerobic (low NADH) vs. anaerobic (high NADH) conditions. It is difficult to determine the dose-response function of the biosensor (i.e., reporter fluorescence over a range of NADH/NAD⁺ ratios) because the NAD(H) levels cannot be directly determined by exogenous supplementation in the growth media. However, based on end-point measurements, the dynamic range and maximum reporter signal were determined. The FMN-based fluorescent protein (FbFP) was used as a reporter instead of GFP because FbFP is active in anaerobic conditions. Cells constitutively expressing the reporter (no pB-Rex) had comparable fluorescence in aerobic and anaerobic conditions (FIGS. 5 and 6, left bar of each set). This result suggested that promoter strength is the same under aerobic and anaerobic growth and that any differences in fluorescence observed when B-Rex is co-expressed were primarily dependent on B-Rex activity. It also established that the strength of the constitutive promoter was not impaired by insertion of operator sites or from minor differences in ROP-PP and ROP-IP sequences. Under aerobic conditions (low NADH), when B-Rex was co-expressed, reporter fluorescence was diminished by 30- and 10-fold, respectively, in pROP-PP and pROP-IP containing cells (FIGS. 5 and 6, compare ‘Aerobic’ left and right bars). This result was consistent with the expectation that B-Rex should exert stronger repression by binding tighter to the perfect than the imperfect palindrome. Under anaerobic conditions (high NADH), both promoters were activated and their dynamic range, computed as the ratio of reporter fluorescence in anaerobic (high NADH) and aerobic (low NADH) conditions, was similar: pROP-PP, 4.9-fold and pROP-IP, 4.0-fold (FIGS. 5 and 6, compare ‘Aerobic’ and ‘Anaerobic’ right bars). This is consistent with the 2.5-fold difference observed using the enzyme-based assay (FIG. 7). Maximum reporter signal at induction was well below constitutive reporter expression (FIGS. 5 and 6, compare ‘Anaerobic’ left and right bars) suggesting that the biosensor was not saturated and that the physiological range of NADH/NAD⁺ ratio under these growth conditions was within the detection range of both B-Rex promoter designs. Although the dynamic range of pROP-PP and pROP-IP promoters were similar, the induced reporter signal was much higher with pROP-IP than pROP-PP (13695 fluorescence units vs. 4509 fluorescence units, FIGS. 5 and 6). Therefore, the ROP-IP system was used for further experiments in this study. GFP was used as the reporter in the following experiments as they were carried out under aerobic conditions.

### Example 3: Redox State in Respiratory Chain Mutants

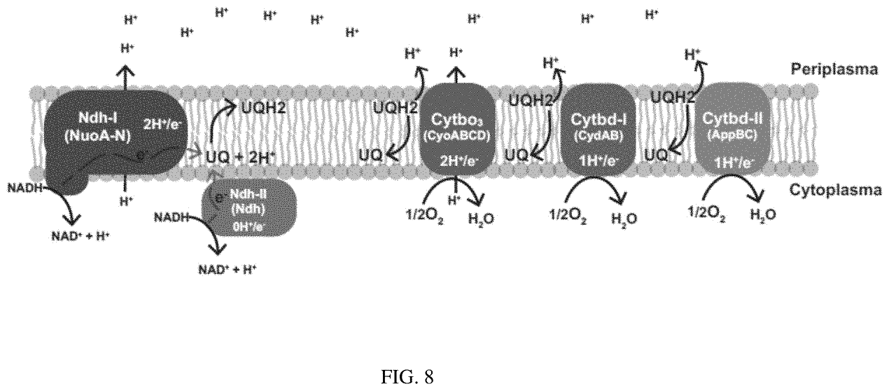

NADH metabolism is closely linked to energy production due to its role in ATP synthesis. The E. coli respiratory chain is comprised of membrane-bound NADH dehydrogenases and cytochrome oxidases (FIG. 8). NADH dehydrogenases oxidize NADH and transfer electrons to quinones, which are subsequently oxidized by cytochrome oxidases passing electrons to the terminal electron acceptor O₂ to make H₂O. The respiratory chain converts energy stored in NADH to proton gradients, which can be used to power ATP synthesis (FIG. 8). In E. coli, there are two major NADH dehydrogenases, Ndh-I and Ndh-II. Ndh-I or NADH:ubiquinone oxidoreductase I (NuoA-N) is a multi-subunit enzyme, which is similar in sequence to complex I of other bacteria, archaea, and eukaryotes. Ndh-II or NADH:quinone oxidoreductase II is a single-subunit protein present in microbes but not in human mitochondria. Ndh-I couples energy released by NADH oxidation to pumping protons, but Ndh-II only catalyzes oxidation of NADH and reduction of quinones without the ability to pump protons. In E. coli, there are three respiratory chain cytochrome oxidases: Cytbo₃ (CyoABCD), Cytbd-I (CydAB) and Cytbd-II (AppBC) and these enzymes are suggested to function under different conditions. Since most cellular NADH is consumed in aerobic respiration, it was hypothesized that mutating respiratory chain genes would likely alter NADH/NAD⁺ ratio.

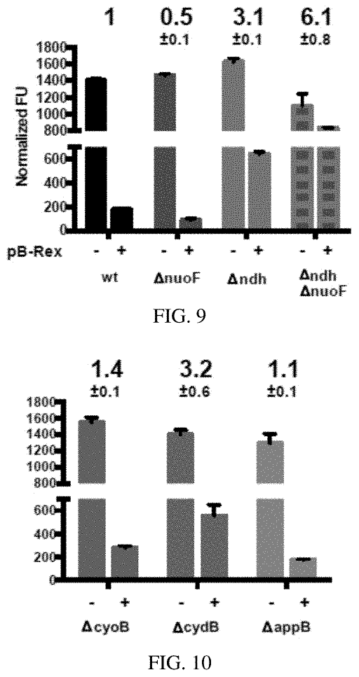

To study the effects of NADH dehydrogenases on NADH/NAD⁺ ratio, the F-subunit of Ndh-I (ΔnuoF) and Ndh-II (Δndh) were knocked out individually and both together (ΔnuoFAndh). The F-subunit of Ndh-I is directly involved in FMN-mediated oxidation of NADH. The Δndh strain gave 3.1-fold higher reporter fluorescence relative to wild-type E. coli (FIG. 9). This is consistent with previous studies reporting Ndh-II as the predominant pathway for NADH oxidation in E. coli. However, the reporter fluorescence of ΔnuoF strain was half of wild type E. coli. This result was unexpected, but consistently observed when experiments were repeated. However, without determining the lower sensitivity threshold of the biosensor, it is not known if that actual NADH/NAD⁺ change is significant. One plausible mechanistic explanation based on prior art studies, is that knocking out Ndh-I partially uncouples NADH oxidation from proton-pumping. Therefore, to maintain the desired proton gradient, cells may increase NADH oxidation through Ndh-II, which might lead to the observed lower NADH/NAD⁺ in ΔnuoF strain. Higher Ndh-II activity would increase the quinol pool, allowing cytochrome oxidases to compensate loss of proton gradient. The double knockout ΔnuoFAndh strain gave the highest reporter fluorescence (6.1-fold higher than wild type), reaching nearly the same level as a constitutively active reporter (FIG. 9). This indicates high accumulation of NADH as expected due to the loss of both NADH dehydrogenases (FIG. 9).

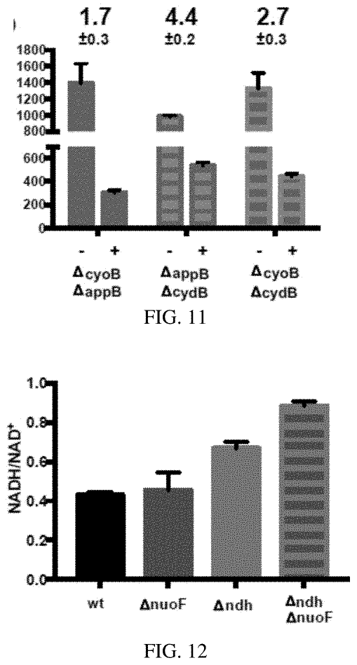

Next, to investigate the impact of cytochrome oxidases Cytbo₃ (cyo), Cytbd-I (cyd) and Cytbd-II (app) on NADH/NAD+ ratio, single and double knockouts of all three enzymes were generated by knocking out the heme-binding subunits CyoB, AppB, CydB of these cytochrome oxidases. Although all three cytochrome oxidases could be knocked out under anaerobic conditions, the cells could not grow aerobically which is consistent with a previous study. Reporter fluorescence of ΔcyoB and ΔappB strains were comparable to wild type, but ΔcydB strain showed 3.2-times greater fluorescence than wild type (FIG. 10). Among the double knockouts, reporter fluorescence of ΔcyoBΔappB was modestly higher (1.7-times) than wild type. However, both double mutants lacking cydB (ΔappBΔcydB and ΔcyoBΔcydB) gave 4.4- and 2.7-fold greater reporter fluorescence than wild type, respectively (FIG. 11). Proton-pumping Cytbo₃ (cyo) is thought to be the major quinol oxidase under aerobic condition, expressed at a level ˜300 molecules per cell. Cytochrome bd-I (cydB) is induced under microaerobic and anaerobic conditions, but is also expressed at a comparable amount ˜200 molecules per cell. The results show that knocking out Cytbd-I in aerobically grown cells significantly increases NADH/NAD⁺ redox ratio suggesting Cytbd-I could play an important role in maintaining redox balance through quinol oxidation even in aerobic conditions. Why E. coli harbors three cytochrome oxidases and the redundancy built into the bacteria respiratory chain is not fully understood. It is apparent from these results that one cytochrome oxidase is sufficient to maintain the redox state in standard laboratory growth conditions using oxygen as electron acceptor. Growth under different conditions could possibly reveal a greater dependency on a given cytochrome oxidase.

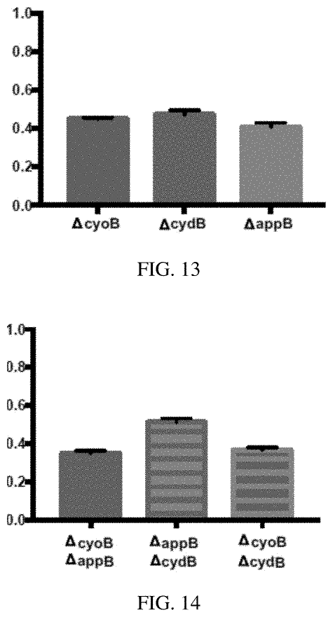

The biosensor-reporter measurements were validated independently for all strains using a commercial enzyme-based NAD(H) quantification system. The results from both methods were in broad agreement (FIGS. 12-14, described in Methods) except for the ΔcydB and ΔnuoF strains. The redox states of these strains were closer to wild type than estimated by the biosensor-reporter. It is likely that the NADH/NAD⁺ ratio may be close to the lower threshold of sensitivity of the biosensor for ΔcydB and ΔnuoF strains. Discrepancy may also arise due reduced sensitivity of the enzyme-based assay caused by a relatively high baseline at low NADH/NAD⁺ levels. However, NADH/NAD⁺ trends determined by the enzymatic assay were generally consistent with reporter fluorescence.

### Example 4: Redox State in E. coli Grown Under Different Carbon Sources

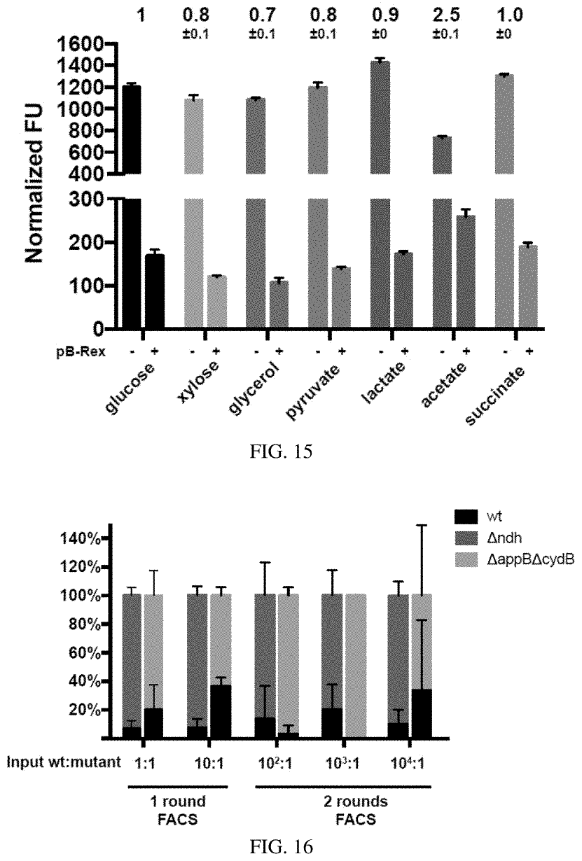

E. coli is well known for its flexible metabolism, which allows it to grow on different nutrition sources. Since each source activates metabolic pathways with different redox demands, the biosensor-reporter was used to measure NADH/NAD⁺ levels in E. coli cells grown aerobically with several common carbon sources. Glucose (as a reference), glycerol, pyruvate, glycerol, acetate, xylose, and succinate were used. Glycerol, pyruvate and lactate feed directly into glycolysis, succinate is an important TCA cycle intermediate; xylose is a five-carbon sugar metabolized through pentose phosphate pathway before entering glycolysis. Acetate metabolism is more complex requiring the glyoxylate shunt along with other essential pathways to support growth. It was observed that reporter fluorescence in nearly all carbon sources was slightly below glucose (FIG. 15) suggesting that NADH/NAD⁺ is modestly altered by changing carbon source. However, cells grown on acetate gave 2.5-times higher reporter fluorescence than glucose. This result is consistent with a detailed prior art metabolomic study where E. coli grown on acetate exhibited an NADH/NAD⁺ ratio twice that of glucose: approximately 0.055 in acetate compared to approximately 0.03 in glucose. Acetate is a fermentation product of E. coli that can be secreted at elevated intracellular NADH/NAD⁺ when glucose uptake surpasses the metabolic capacity of the cells in aerobic growth. Acetate is also known to cause global stress responses and inhibit cell growth. It is unclear if elevated NADH/NAD⁺ we observe is required to assimilate acetate for growth or is a result of a stress response caused by acetate. The biosensor is useful for comparing relative differences in NADH/NAD⁺ because change in reporter fluorescence is proportional to NADH/NAD+ ratio, but the numerical value of fold change in reporter may not be the same as fold change in NADH/NAD⁺. Quantitative comparisons of NADH/NAD⁺ based on reporter fluorescence requires a calibration curve, which would be hard to establish due to challenges with exogenous supplementation of NADH in the growth medium (rapid oxidation and poor transport). However, with advances in cell-free platforms for transcription-based reporting, this may soon be possible.

### Example 5: Redox Biosensor-Reporter System as a Tool for High-Throughput Screening

Engineering microbes for production of fuels and chemicals has largely focused on optimizing enzyme levels to facilitate high carbon flux through a desired pathway. However, the cofactor ratio is increasingly recognized as a major bottleneck because altering the ratio of reduced to oxidized forms can provide the thermodynamic driving force for less favorable reactions. For instance, availability of excess reducing equivalents improved n-butanol productivity to a very high yield. High-throughput screening is a powerful tool for identifying strain backgrounds with altered NADH/NAD⁺ pool, a task that can otherwise be very difficult by conventional analytical methods. Biosensors have been used with great success to evolve and engineer microbes by high-throughput screening.

To evaluate if the redox biosensor-reporter could be used for screening, E. coli mutants were enriched with high NADH/NAD+ from a mixed population with wild-type E. coli. To mimic a library of mutants with varying proportions of high NADH variants, wild-type E. coli was mixed with either Δndh or ΔcydBΔappB mutants in the following proportions: 1:1, 10:1, 1,000:1, 10,000:1. Δndh and ΔcydBΔappB strains gave 3.1- and 4.4-fold higher NADH/NAD+ signal, respectively, compared to wildtype (FIGS. 9 and 11). Using fluorescence activated cell sorting, the top 5% fluorescent cells from each mixture were sorted, plated, and 30 randomly chosen clones were genotyped as wildtype or mutant by PCR. For higher dilutions (100:1 to 10,000:1 of mutant to wild-type E. coli), the sorted samples were grown to exponential phase and top 5% fluorescent cells of the regrown sample were sorted again, followed by plating and colony PCR. The results show that even at the highest proportion of wildtype to mutant (10,000:1) nearly 80% of the clones after enrichment were mutants (FIG. 16). This result suggests that the redox biosensor-reporter can be used to screen medium-sized pooled libraries containing up to 10⁴ variants.

The large error in the 10,000:1 wildtype-to-ΔcydBΔappB test suggests that it is close to the limit of detection of the biosensor-reporter. To assess if the mutants have a growth advantage over wild type, the growth rate of all three strains was measured individually in a plate reader. The mutants grew slower than wild type and were therefore, if anything, at a minor growth disadvantage. Since regrowth of sorted cells may distort the population distribution, the fraction of mutants recovered after sorting may be confounded by artifacts due to sorting. In summary, the redox biosensor-reporter can be used to screen medium-sized strain libraries containing up to 10⁴ variants for altered NADH capacity. More sensitive screens can be carried out by enhancing the dynamic range of the biosensor-reporter by further promoter engineering and/or by additional rounds of enrichment by cell sorting.

### Discussion

A redox biosensor system based on transcription repressor Rex for accurate, non-invasive measurement of NADH/NAD⁺ ratio in bacteria was constructed and validated. By placing operator sites of varying affinities within a constitutive E. coli promoter, the dynamic range and maximum reporter signal of Rex can be optimized to make it a useful regulatory biosensor. The Rex biosensor coupled to a reporter proved to be an effective tool to screen variant libraries based on redox and could also enable dynamic control of metabolic pathways in response to intracellular redox changes. Since Rex is a repressor, it could be ported to non-model bacteria with known constitutive promoter for redox sensing by optimizing the placement of its operator site. Rex-based biosensors could potentially be used in eukaryotes by fusing to the Rex protein domains that activate or repress transcription (e.g., VP16 or KRAB) or that modify the epigenetic state in response to redox changes.

The redox differences observed in the respiratory chain mutants highlight the complex underlying relationship between cofactor metabolism, cellular redox, and respiration. When Ndh-I is knocked out, NADH oxidation step may be uncoupled from proton-pumping, resulting in a loss of proton gradient in the first half of the respiratory chain. To maintain the desired proton gradient for numerous cellular processes, cells may upregulate NADH oxidation to reduce more quinone to allow cytochrome oxidases to compensate the loss of proton gradient. It has been observed increased NADH-coenzyme Q1 reductase activity in membrane extracts from an Ndh-I deletion (ΔnuoF) strain than wild-type, suggesting an upregulation in NADH oxidation in the Ndh-I mutant. It was also found that when Ndh-I and -II were knocked out, NADH oxidation in membrane extracts was fully abolished, which is consistent with the observation of highest NADH/NAD+ in ΔnuoFAndh double mutant. Under surplus nutrition, as is the case in glucose-unlimited media experiments, although both Ndh-I and Ndh-II may be active, the uncoupled NADH dehydrogenase Ndh-II may be dominant because cells need to counteract increasing NADH/NAD+ triggered by faster metabolism. As glucose uptake increases, E. coli is reported to upregulate Ndh-II expression more than Ndh-I expression to compensate for increasing NADH/NAD⁺. This could be because proton-coupled NADH dehydrogenase Ndh-I is calibrated to oxidize NADH to maintain the right proton motive force required for ATP synthesis. In contrast, under glucose-limiting condition, it was observed that knocking out Ndh-II (Δndh) had only a small effect on respiration, leading to the conclusion that Ndh-I is the main driver of NADH oxidation. Based on these results and on previous reports, it is hypothesized that cells might adjust NADH oxidation through Ndh-I and Ndh-II under different growth conditions. When carbon source is unlimited, Ndh-II oxidizes excess NADH to support redox balance and fast growth, and when favored carbon source like glucose becomes limited, cells may increase energy efficiency to favor NADH oxidation by Ndh-I as a mechanism to conserve energy and generate proton motive force for ATP synthesis. The redox biosensor could be useful tool to gain a deeper mechanistic understanding of the complex genetic and environmental causes affecting cellular redox.

High-throughput redox screening has potential utility to increase understanding of microbial metabolism with applications in both medicine and biotechnology. Linking redox changes to mutations on a genome-wide scale provides a comprehensive genotype-phenotype metabolic map of an organism, leading to a better understanding of redox flow and metabolism. Traditional genome-wide screens generally require a life or death selection to enrich or deplete mutants with the desired phenotype. However, redox changes cannot easily be tied directly to cell survival. A redox biosensor enables genome-wide screening by sorting and deep sequencing barcoded mutant libraries into fluorescent bins. Microbial screens using the Rex biosensor described herein can be used in several applications. A genome-wide redox screen could identify mutations that perturb the redox state and affect cell viability, leading to valuable insight into new mechanisms by which antibiotic resistance may emerge. It has been shown that bactericidal antibiotics, such as gyrase or peptidoglycan inhibitors, kill by a common hydroxyl radical mechanism mediated by a large drop in NADH/NAD⁺ ratio and knocking out TCA cycle genes to throttle NADH level reduces antibiotic susceptibility. Engineering microbes for producing biofuels or other highly reduced compounds requires excess NADH reducing equivalents to provide the required driving force. Since cells tolerate only a narrow margin of redox imbalance, the lack of reducing power from the cell's native metabolic capacity is often a limitation in pathway optimization. Using the redox biosensor, a microbe can be evolved, through rounds of genome-wide mutagenesis and selection, toward higher reducing capacity, together with a ‘sink’ reaction to consume NADH to maintain overall redox balance.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A redox biosensor system for measurement of NADH/NAD⁺ ratio, comprising
a host strain or cell-free system comprising a first expression cassette and a second expression cassette,
the first expression cassette comprising, in operable communication, a constitutive promoter and a gene encoding a Rex allosteric transcription factor that regulates gene expression by binding to NAD(H), and
the second expression cassette comprising, in operable communication, a promoter regulated by the Rex allosteric transcription factor, and a gene encoding a reporter protein,
wherein the first expression cassette expresses the Rex allosteric transcription factor, and, in the presence of NAD(H), the Rex allosteric transcription factor drives expression of the reporter protein from the second expression cassette, and wherein the expression of the reporter protein is proportional to the NADH/NAD⁺ ratio.

2. The redox biosensor system of claim 1, wherein the biosensor is functional under hypoxic, aerobic or anaerobic conditions.

3. The redox biosensor system of claim 1, wherein the constitutive promoter in the first expression cassette comprises any one or more of SEQ ID NOs. 2-8.

4. The redox biosensor system of claim 1, wherein the promoter regulated by the Rex allosteric transcription factor comprises a modified constitutive promoter comprising one or more Rex operator sites.

5. The redox biosensor system of claim 1, wherein the first expression cassette and the second expression cassette are present on the same or different plasmids.

6. The redox biosensor system of claim 1, wherein the first expression cassette is present in a high or low copy number sensor plasmid, and the second expression cassette is present on a reporter plasmid.

7. The redox biosensor system of claim 1, wherein the Rex allosteric transcription factor is B. subtilis Rex.

8. The redox biosensor system of claim 7, wherein the promoter regulated by the Rex allosteric transcription factor comprises TGTGAAATACTTCACA (SEQ ID NO: 48; ROP-PP) or TGTGAAATATTGAGCA (SEQ ID NO: 49; ROP-IP).

9. The redox biosensor system of claim 1, wherein the host strain comprises organisms of the genus Escherichia, Bacillus, Staphylococcus, Caulobacter, Streptococcus, Thermus, Streptomyces, Mycoplasma, Aliivibrio, Synechocystis, Azotobacter, Pseudomonas, Agrobacterium, Zymomonas, or a combination of the foregoing

10. The redox biosensor system of claim 1, wherein the host strain comprises E. coli.

11. The redox biosensor system of claim 1, wherein the reporter protein comprises GFP or FbFP.

12. A liquid-based assay, a paper strip, or a multi-well plate assay comprising the redox biosensor system of claim 1.

13. A method of determining NADH/NAD⁺ ratio, comprising contacting a sample suspected of containing NADH/NAD⁺ with the redox biosensor system of claim 1.

14. The method of claim 13, wherein the sample is an engineered microbe.

15. The method of claim 14, wherein the engineered microbe produces a biofuel or a chemical.

16. The method of claim 13, wherein the sample is a cell sample to be assayed for a disorder associated with metabolism.

17. The method of claim 16, wherein the disorder associated with metabolism is cancer or an autoimmune disease.

18. A method of screening a population of engineered microbes for a target NADH/NAD⁺ ratio, comprising
contacting the population of engineered microbes with the redox biosensor system of claim 1, and
identifying a sub-population of engineered microbes having the target NADH/NAD⁺.

19. The method of claim 18, further comprising
contacting the sub-population of engineered microbes having the target NADH/NAD⁺ ratio with the redox biosensor system of claim 1,
identifying a further sub-population of engineered microbes having the target NADH/NAD⁺ ratio, and
optionally repeating the contacting and identifying for a plurality of rounds to provide an optimized sub-population of engineered microbes having the target NADH/NAD⁺ ratio.

20. The method of claim 18, further comprising deep sequencing the sub-population of engineered microbes having the target NADH/NAD⁺ ratio.

21. The method of claim 20, further comprising identifying one or more genes in the sub-population of engineered microbes that perturb redox state.


SEQUENCE LISTING (50 sequences)

SEQ ID NO: 1 - protein, 215 aa, Bacillus subtilis
MNKDQSKIPQATAKRLPLYYRFLKNLHASGKQRVSSAELSDAVKVDSATIRRDFSYFGALGKKGYGYNVDYLLSFFRKTLDQDEMTDVILIGVGNLGTAFLHYNFTKNNNTKISMAFDINESKIGTEVGGVPVYNLDDLEQHVKDESVAILTVPAVAAQSITDRLVALGIKGILNFTPARLNVPEHIRIHHIDLAVELQSLVYFLKHYSVLEEIE

SEQ ID NO: 2 - DNA, 30 bp, Escherichia coli
agctagctcagtcctaggtattatgctagc

SEQ ID NO: 3 - DNA, 50 bp, Escherichia coli
ggcgcgccttgacataaagtctaacctataggatacttacagccatacaa

SEQ ID NO: 4 - DNA, 48 bp, Escherichia coli
ggcgcgccttgacggctagctcagtcctaggtacagtgctagcttaat

SEQ ID NO: 5 - DNA, 48 bp, Escherichia coli
ggcgcgccttgacaattaatcatccggctcctaggatgtgtggaggga

SEQ ID NO: 6 - DNA, 49 bp, Escherichia coli
ggcgcgccttgtattttaatcatccggctcgtataatgtgtggaatcca

SEQ ID NO: 7 - DNA, 47 bp, Escherichia coli
ggcgcgccttgacagctagctcagtcctaggtataatgctagcacga

SEQ ID NO: 8 - DNA, 57 bp, Escherichia coli
ggcgcgcccacggtgttagacatttatcccttgcggcgatataatgtgtggatccat

SEQ ID NO: 9 - DNA, 40 bp, Artificial Sequence
  Primer
aggcgcgcccgggatctcgacgctctcccttatgagatct

SEQ ID NO: 10 - DNA, 20 bp, Artificial Sequence
  Primer
gcttgatatcgaattcctgc

SEQ ID NO: 11 - DNA, 45 bp, Artificial Sequence
  Primer
cagacgattctattacaaatccagaatgaataaggatcaatcaaa

SEQ ID NO: 12 - DNA, 47 bp, Artificial Sequence
  Primer
cctaggatgtgtggagggacgactaacaccagacgattctattacaa

SEQ ID NO: 13 - DNA, 60 bp, Artificial Sequence
  Primer
tcgagatcccgggcgcgccttgacaattaatcatccggctcctaggatgtgtggagggac

SEQ ID NO: 14 - DNA, 40 bp, Artificial Sequence
  Primer
aatctattaggaggtttatagcatggcgtcctttcagtct

SEQ ID NO: 15 - DNA, 40 bp, Artificial Sequence
  Primer
aggaattcgatatcaagcttttactccaacagcttctcgt

SEQ ID NO: 16 - DNA, 25 bp, Artificial Sequence
  Primer
aagcttgatatcgaattcctgcagc

SEQ ID NO: 17 - DNA, 22 bp, Artificial Sequence
  Primer
gctagcatctcgaggtgaagac

SEQ ID NO: 18 - DNA, 25 bp, Artificial Sequence
  Primer
ccctttttctttaaaaccgaaaaga

SEQ ID NO: 19 - DNA, 55 bp, Artificial Sequence
  Primer
tggagcaagaaggtaagcgaggataagaaggaaaagaatggtttccaagggcgag

SEQ ID NO: 20 - DNA, 55 bp, Artificial Sequence
  Primer
ctttacggctagctcagccctaggtattatgctagcatggagcaagaaggtaagc

SEQ ID NO: 21 - DNA, 45 bp, Artificial Sequence
  Primer
tcggttttaaagaaaaagggggcgcgcctttacggctagctcagc

SEQ ID NO: 22 - DNA, 40 bp, Artificial Sequence
  Primer
ggcaaggtgtcaccaccctgttatttgtacagctcatcca

SEQ ID NO: 23 - DNA, 40 bp, Artificial Sequence; identical to SEQ ID NO: 22
  Primer

SEQ ID NO: 24 - DNA, 40 bp, Artificial Sequence
  Primer
gatgctagcaaaaaaagagtatttacagctagctcagtcc

SEQ ID NO: 25 - DNA, 35 bp, Artificial Sequence
  Primer
tttcgtcttcacctcgagatgctagcaaaaaaaga

SEQ ID NO: 26 - DNA, 40 bp, Artificial Sequence
  Primer
tttataagaattgtgaattctttttgtgaagtatttcaca

SEQ ID NO: 27 - DNA, 60 bp, Artificial Sequence
  Primer
tttacagctagctcagtcctaggtattattacagccatgtgaaatacttcacaaaaagaa

SEQ ID NO: 28 - DNA, 40 bp, Artificial Sequence; identical to SEQ ID NO: 26
  Primer

SEQ ID NO: 29 - DNA, 53 bp, Artificial Sequence
  Primer
tttacagctagctcagtcctaggtattattacagccatgtgaaatattgagca

SEQ ID NO: 30 - DNA, 38 bp, Artificial Sequence
  Primer
tataagaattgtgaattctttttgctcaatatttcaca

SEQ ID NO: 31 - DNA, 30 bp, Artificial Sequence
  Primer
gaattcattaaagaggagaaaggtaccatg

SEQ ID NO: 32 - DNA, 22 bp, Artificial Sequence
  Primer
gctagcatctcgaggtgaagac

SEQ ID NO: 33 - DNA, 55 bp, Artificial Sequence; identical to SEQ ID NO: 19
  Primer

SEQ ID NO: 34 - DNA, 55 bp, Artificial Sequence; identical to SEQ ID NO: 20
  Primer

SEQ ID NO: 35 - DNA, 45 bp, Artificial Sequence; identical to SEQ ID NO: 21
  Primer

SEQ ID NO: 36 - DNA, 40 bp, Artificial Sequence; identical to SEQ ID NO: 22
  Primer

SEQ ID NO: 37 - DNA, 40 bp, Artificial Sequence
  Primer
tggatgagctgtacaaataacagggtggtgacaccttgcc

SEQ ID NO: 38 - DNA, 40 bp, Artificial Sequence; identical to SEQ ID NO: 24
  Primer

SEQ ID NO: 39 - DNA, 35 bp, Artificial Sequence; identical to SEQ ID NO: 25
  Primer

SEQ ID NO: 40 - DNA, 40 bp, Artificial Sequence; identical to SEQ ID NO: 26
  Primer

SEQ ID NO: 41 - DNA, 53 bp, Artificial Sequence; identical to SEQ ID NO: 29
  Primer

SEQ ID NO: 42 - DNA, 38 bp, Artificial Sequence; identical to SEQ ID NO: 30
  Primer

SEQ ID NO: 43 - DNA, 16 bp, Artificial Sequence
  Operator palindrome
  misc_feature (7)..(10): n is a, c, g, or t
tgtgaannnnttcaca

SEQ ID NO: 44 - DNA, 16 bp, Artificial Sequence
  Operator palindrome
  misc_feature (7)..(10): n is a, c, g, or t
tgtgaannnntgcaca

SEQ ID NO: 45 - DNA, 16 bp, Artificial Sequence
  Operator palindrome
  misc_feature (7)..(10): n is a, c, g, or t
tgtgaannnntgatca

SEQ ID NO: 46 - DNA, 17 bp, Artificial Sequence
  Operator palindrome
  misc_feature (7)..(10): n is a, c, g, or t
tgtgaannnntgatgca

SEQ ID NO: 47 - DNA, 16 bp, Artificial Sequence
  Operator palindrome
tgtgaaatattgagca

SEQ ID NO: 48 - DNA, 16 bp, Artificial Sequence
  Operator palindrome
tgtgaaatacttcaca

SEQ ID NO: 49 - DNA, 16 bp, Artificial Sequence
  Operator palindrome
tgtgaaatattgagca

SEQ ID NO: 50 - DNA, 16 bp, Artificial Sequence
  Operator palindrome
  misc_feature (7)..(10): n is a, c, g, or t
tgctcannnntgagca